(19) United States
(12) Patent Application Publication
(10) Pub. No.: US 2025/0352032 A1
(43) Pub. Date: Nov. 20, 2025

(54) MEDICAL DEVICE, MEDICAL SYSTEM, LEARNING DEVICE, METHOD OF OPERATING MEDICAL DEVICE, AND COMPUTER-READABLE RECORDING MEDIUM

(71) Applicant: OLYMPUS MEDICAL SYSTEMS CORP., Tokyo (JP)

(72) Inventors: Yasuo TANIGAMI, Tokyo (JP); Yusuke OTSUKA, Yokohama-shi (JP); Noriko KURODA, Tokyo (JP); Takaaki IGARASHI, Tokyo (JP)

(73) Assignee: OLYMPUS MEDICAL SYSTEMS CORP., Tokyo (JP)

(21) Appl. No.: 19/289,335

(22) Filed: Aug. 4, 2025

Related U.S. Application Data
(63) Continuation of application No. PCT/JP2023/004405, filed on Feb. 9, 2023.

(51) Int. Cl. A61B 1/00; A61B 1/04; A61B 1/06; G06T 7/00; G16H 30/40

(52) U.S. Cl. CPC A61B 1/000096; A61B 1/00045; A61B 1/043; A61B 1/0638; A61B 1/0661; G06T 7/0014; G16H 30/40; G06T 2207/10064; G06T 2207/10068; G06T 2207/20081; G06T 2207/30096

(57) ABSTRACT

A medical device includes a processor including hardware, the processor being configured to acquire setting information in which a region of interest is set for a biological tissue and thermal denaturation information regarding a thermally denatured region in which thermal denaturation has occurred by heat treatment for the biological tissue, determine whether or not there is the thermally denatured region outside the region of interest based on the setting information and the thermal denaturation information, and output support information indicating that there is the thermally denatured region outside the region of interest when it is determined that there is the thermally denatured region outside the region of interest.

### CROSS REFERENCES TO RELATED APPLICATIONS

This application is a continuation of International Application No. PCT/JP2023/004405, filed on Feb. 9, 2023, the entire contents of which are incorporated herein by reference.

### BACKGROUND

### 1. Technical Field

The present disclosure relates to a medical device, a medical system, a learning device, a method of operating a medical device, and a computer-readable recording medium.

### 2. Related Art

Hitherto, in the medical field, a technology for visualizing a state of cauterization of a subject such as a biological tissue using an energy device or the like is known (see, for example, WO 2020/054723 A). In the technology, the subject is irradiated with excitation light, and an image and information including fluorescence image data generated based on an imaging signal acquired by imaging fluorescence generated from a thermally invasive region of the subject by receiving the excitation light are displayed, thereby visualizing the cauterization state for a user such as an operator.

### SUMMARY

In some embodiments, a medical device includes a processor including hardware, the processor being configured to acquire setting information in which a region of interest is set for a biological tissue and thermal denaturation information regarding a thermally denatured region in which thermal denaturation has occurred by heat treatment for the biological tissue, determine whether or not there is the thermally denatured region outside the region of interest based on the setting information and the thermal denaturation information, and output support information indicating that there is the thermally denatured region outside the region of interest when it is determined that there is the thermally denatured region outside the region of interest.

In some embodiments, a medical system includes: a light source device; an imaging device; and a medical device. The light source device includes a special light source configured to generate special light for a biological tissue; and an excitation light source configured to generate excitation light that excites advanced glycation end products generated by performing heat treatment on the biological tissue, the imaging device includes an imaging element configured to generate an imaging signal by imaging return light or light emitted from the biological tissue irradiated with the special light or the excitation light, the medical device includes a processor, and the processor is further configured to acquire setting information in which a region of interest is set for the biological tissue and thermal denaturation information regarding a thermally denatured region in which thermal denaturation has occurred by the heat treatment for the biological tissue, determine whether or not there is the thermally denatured region outside the region of interest based on the setting information and the thermal denaturation information, and output support information indicating that there is the thermally denatured region outside the region of interest when it is determined that there is the thermally denatured region outside the region of interest.

In some embodiments, a learning device includes a processor including hardware, the processor being configured to generate a trained model by performing machine learning using training data in which a plurality of fluorescence images generated based on an imaging signal generated by imaging light emitted from a thermally denatured region by irradiating a biological tissue with excitation light and a plurality of white light images generated based on an imaging signal generated by imaging return light by irradiating the biological tissue with white light are input data, and support information indicating that there is a thermally denatured region included in the fluorescence image outside a region of interest included in each of the plurality of white light images is output data.

In some embodiments, provided is a method of operating a medical device including a processor. The method includes: acquiring, by the processor, setting information in which a region of interest is set for a biological tissue and thermal denaturation information regarding a thermally denatured region in which thermal denaturation has occurred by heat treatment for the biological tissue; determining, by the processor, whether or not there is a thermally denatured region outside the region of interest based on the setting information and the thermal denaturation information; and outputting, by the processor, support information indicating that there is a thermally denatured region outside the region of interest when it is determined that there is the thermally denatured region outside the region of interest.

In some embodiments, provided is a non-transitory computer-readable recording medium with an executable program stored thereon. The program causes the processor of a medical device to execute: acquiring setting information in which a region of interest is set for a biological tissue and thermal denaturation information regarding a thermally denatured region in which thermal denaturation has occurred by heat treatment for the biological tissue; determining whether or not there is the thermally denatured region outside the region of interest based on the setting information and the thermal denaturation information; and outputting support information indicating that there is the thermally denatured region outside the region of interest when it is determined that there is the thermally denatured region outside the region of interest.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a configuration of an endoscope system according to a first embodiment;

FIG. 2 is a block diagram illustrating a functional configuration of a main part of the endoscope system according to the first embodiment;

FIG. 3 is a diagram schematically illustrating wavelength characteristics of excitation light emitted by two light source units according to the first embodiment;

FIG. 4 is a diagram schematically illustrating a configuration of a pixel unit according to the first embodiment;

FIG. 5 is a diagram schematically illustrating a configuration of a color filter according to the first embodiment;

FIG. 6 is a diagram schematically illustrating sensitivity and a wavelength band of each filter according to the first embodiment;

FIG. 7A is a diagram schematically illustrating signal values of G pixels of an imaging element according to the first embodiment;

FIG. 7B is a diagram schematically illustrating signal values of R pixels of the imaging element according to the first embodiment;

FIG. 7C is a diagram schematically illustrating signal values of B pixels of the imaging element according to the first embodiment;

FIG. 8 is a diagram schematically illustrating a configuration of a cut filter according to the first embodiment;

FIG. 9 is a diagram schematically illustrating a transmission characteristic of the cut filter according to the first embodiment;

FIG. 10 is a diagram schematically illustrating a transmission characteristic of the cut filter according to the first embodiment;

FIG. 11 is a flowchart illustrating an outline of processing executed by a control device 9 according to the first embodiment;

FIG. 12 is a diagram schematically illustrating a region of interest set for a first image by a setting unit 953 according to the first embodiment;

FIG. 13 is a diagram schematically illustrating a thermally denatured region specified for a second image by a specification unit 954 according to the first embodiment;

FIG. 14 is a diagram schematically illustrating alignment processing executed by an alignment unit 955 according to the first embodiment;

FIG. 15 is a flowchart illustrating an outline of processing executed by a control device 9 according to a second embodiment;

FIG. 16 is a diagram illustrating a schematic configuration of an endoscope system according to a third embodiment;

FIG. 17 is a flowchart illustrating an outline of processing executed by a control device 9 according to the third embodiment;

FIG. 18 is a diagram illustrating a schematic configuration of an endoscope system according to a fourth embodiment;

FIG. 19 is a block diagram illustrating a functional configuration of a medical device 13 according to the fourth embodiment; and

FIG. 20 is a diagram illustrating a functional configuration of a main part of an endoscope system 1C according to a fifth embodiment.

### DETAILED DESCRIPTION

Hereinafter, modes for carrying out the present disclosure will be described in detail with reference to the drawings. Note that the present disclosure is not limited to the following embodiments. In addition, each drawing referred to in the following description merely schematically illustrates a shape, a size, and a positional relationship to the extent that the content of the present disclosure can be understood. That is, the present disclosure is not limited only to the shape, the size, and the positional relationship illustrated in each drawing. Further, in the description of the drawings, the same reference signs denote the same parts. Furthermore, as an example of an endoscope system according to the present disclosure, an endoscope system including a rigid endoscope and a medical imaging device will be described.

### First Embodiment

### Configuration of Endoscope System

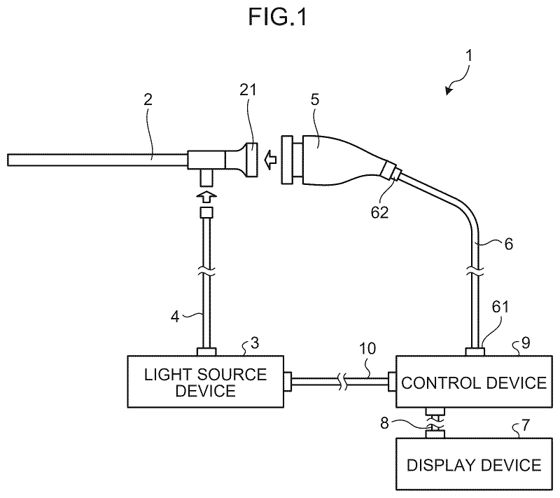

FIG. 1 is a diagram illustrating a configuration of an endoscope system according to a first embodiment. An endoscope system 1 illustrated in FIG. 1 is a system that is used in a medical field and observes and treats a biological tissue in a subject such as a living body. In the first embodiment, a rigid endoscope system using a rigid endoscope (insertion unit 2) illustrated in FIG. 1 will be described as the endoscope system 1, but the present disclosure is not limited thereto, and for example, an endoscope system including a flexible endoscope may be used. Furthermore, an endoscope system can also be applied as the endoscope system 1 to a medical microscope, a medical surgical robot system, or the like that includes a medical imaging device that images a subject and performs surgery, treatment, or the like while displaying an observation image based on an imaging signal (image data) captured by the medical imaging device on a display device.

In addition, in recent years, in the medical field, minimally invasive treatment using an endoscope, a laparoscope, or the like has been widely performed. For example, as the minimally invasive treatment using an endoscope, a laparoscope, or the like, endoscopic submucosal dissection (ESD), laparoscopy and endoscopy cooperative surgery (LECS), non-exposed endoscopic wall-inversion surgery (NEWS), transurethral resection of the bladder tumor (TUR-bt), or the like is widely performed. In the minimally invasive treatment, for example, in order to mark a region to be operated as pretreatment when performing treatment, an operator such as a doctor performs resection by cauterization, marking treatment by heat treatment, or the like on a region of interest (pathogenic region) having an affected part in the biological tissue using a treatment tool of an energy device that emits high-frequency, ultrasonic, or microwave energy. In addition, also in actual treatment, the operator performs treatment such as resection and coagulation of the biological tissue of the subject by using the energy device or the like.

Therefore, the endoscope system 1 illustrated in FIG. 1 is used when performing the surgery or treatment on the subject using the treatment tool (not illustrated) of the energy device or the like capable of performing the heat treatment. Specifically, the endoscope system 1 illustrated in FIG. 1 is used for the transurethral resection of the bladder tumor (TUR-Bt), and is used when performing the treatment on a tumor (bladder cancer) of the bladder or the pathogenic region.

The endoscope system 1 illustrated in FIG. 1 includes the insertion unit 2, a light source device 3, a light guide 4, an endoscope camera head 5 (endoscope imaging device), a first transmission cable 6, a display device 7, a second transmission cable 8, a control device 9, and a third transmission cable 10.

The insertion unit 2 is rigid or at least partially flexible and has an elongated shape. The insertion unit 2 is inserted into the subject such as a patient via a trocar. The insertion unit 2 is provided with an optical system such as a lens that forms the observation image therein.

The light source device 3 is connected to one end of the light guide 4 and supplies illumination light for irradiating the inside of the subject to one end of the light guide 4 under the control of the control device 9. The light source device 3 is implemented by using one or more light sources of any one of semiconductor laser elements such as a light emitting diode (LED) light source, a xenon lamp, and a laser diode (LD), a processor that is a processing device including hardware such as a field programmable gate array (FPGA) and a central processing unit (CPU), and a memory that is a temporary storage area used by the processor. The light source device 3 and the control device 9 may be configured to perform communication individually as illustrated in FIG. 1, or may be integrated with each other.

The light guide 4 has one end detachably connected to the light source device 3, and the other end detachably connected to the insertion unit 2. The light guide 4 guides the illumination light supplied from the light source device 3 from one end to the other end and supplies the illumination light to the insertion unit 2.

An eyepiece portion 21 of the insertion unit 2 is detachably connected to the endoscope camera head 5. The endoscope camera head 5 generates the imaging signal (RAW data) by receiving the observation image formed by the insertion unit 2 and performing photoelectric conversion, and outputs the imaging signal to the control device 9 via the first transmission cable 6 under the control of the control device 9.

The first transmission cable 6 has one end detachably connected to the control device 9 via a video connector 61, and the other end detachably connected to the endoscope camera head 5 via a camera head connector 62. The first transmission cable 6 transmits the imaging signal output from the endoscope camera head 5 to the control device 9, and transmits setting data, power, and the like output from the control device 9 to the endoscope camera head 5. Here, the setting data is a control signal, a synchronization signal, a clock signal, and the like for controlling the endoscope camera head 5.

The display device 7 displays the observation image based on the imaging signal subjected to image processing in the control device 9 and various types of information regarding the endoscope system 1 under the control of the control device 9. The display device 7 is implemented by using a display monitor such as liquid crystal or organic electro luminescence (EL).

The second transmission cable 8 has one end detachably connected to the display device 7, and the other end detachably connected to the control device 9. The second transmission cable 8 transmits the imaging signal subjected to the image processing in the control device 9 to the display device 7.

The control device 9 is implemented by using a processor that is a processing device including hardware such as a graphics processing unit (GPU), an FPGA, or a CPU, and a memory that is a temporary storage area used by the processor. The control device 9 integrally controls operations of the light source device 3, the endoscope camera head 5, and the display device 7 via each of the first transmission cable 6, the second transmission cable 8, and the third transmission cable 10 according to a program recorded in the memory. In addition, the control device 9 performs various types of image processing on the imaging signal input via the first transmission cable 6 and outputs the imaging signal to the second transmission cable 8.

The third transmission cable 10 has one end detachably connected to the light source device 3, and the other end detachably connected to the control device 9. The third transmission cable 10 transmits control data from the control device 9 to the light source device 3.

### Functional Configuration of Main Part of Endoscope System

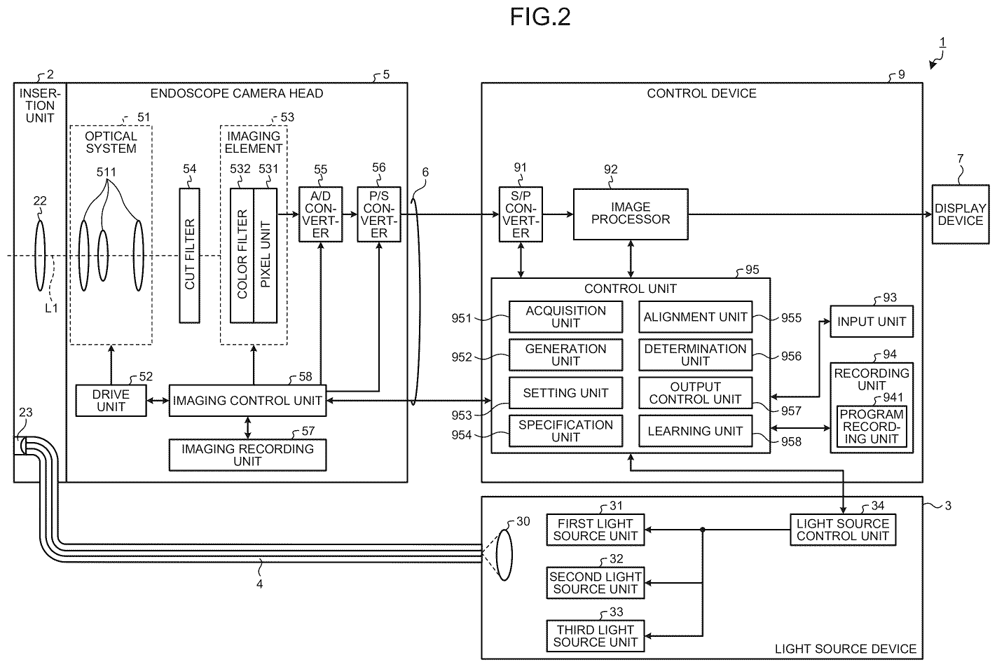

Next, a functional configuration of a main part of the above-described endoscope system 1 will be described. FIG. 2 is a block diagram illustrating the functional configuration of the main part of the endoscope system 1.

### Configuration of Insertion Unit

First, a configuration of the insertion unit 2 will be described. The insertion unit 2 includes an optical system 22 and an illumination optical system 23.

The optical system 22 condenses light such as reflected light reflected from the subject, return light from the subject, excitation light from the subject, and fluorescence emitted from a thermally denatured region thermally denatured by the heat treatment of the energy device or the like to form a subject image. The optical system 22 is implemented by using one or more lenses or the like.

The illumination optical system 23 irradiates the subject with the illumination light supplied from the light guide 4. The illumination optical system 23 is implemented by using one or more lenses or the like.

### Configuration of Light Source Device

Next, a configuration of the light source device 3 will be described. The light source device 3 includes a condenser lens 30, a first light source unit 31, a second light source unit 32, a third light source unit 33, and a light source control unit 34.

The condenser lens 30 condenses light emitted from each of the first light source unit 31, the second light source unit 32, and the third light source unit 33 and emits the light to the light guide 4.

The first light source unit 31 supplies white light (normal light) that is visible light as the illumination light to the light guide 4 by emitting the white light under the control of the light source control unit 34. The first light source unit 31 is implemented using a collimator lens, a white LED lamp, a driver, or the like. The first light source unit 31 may supply the white light that is the visible light by simultaneously performing light emission using a red LED lamp, a green LED lamp, and a blue LED lamp. It is a matter of course that the first light source unit 31 may be implemented using a halogen lamp, a xenon lamp, or the like.

The second light source unit 32 supplies first narrowband light as the illumination light to the light guide 4 by emitting the first narrowband light having a predetermined wavelength band under the control of the light source control unit 34. Here, the first narrowband light has a wavelength band of 530 nm to 550 nm (a central wavelength is 540 nm). The second light source unit 32 is implemented using a green LED lamp, a collimator lens, a transmission filter that transmits light of 530 nm to 550 nm, a driver, or the like.

The third light source unit 33 supplies second narrowband light as the illumination light to the light guide 4 by emitting the second narrowband light having a wavelength band different from that of the first narrowband light under the control of the light source control unit 34. Here, the second narrowband light has a wavelength band of 400 nm to 430 nm (a central wavelength is 415 nm). The third light source unit 33 is implemented by using a semiconductor laser such as a collimator lens or a violet laser diode (LD), a driver, or the like. In the first embodiment, the second narrowband light functions as the excitation light that excites advanced glycation end products generated by performing the heat treatment on the biological tissue.

The light source control unit 34 is implemented by using a processor that is a processing device including hardware such as an FPGA or a CPU, and a memory that is a temporary storage area used by the processor. The light source control unit 34 controls a light emission timing, a light emission time, and the like of each of the first light source unit 31, the second light source unit 32, and the third light source unit 33 based on the control data input from the control device 9.

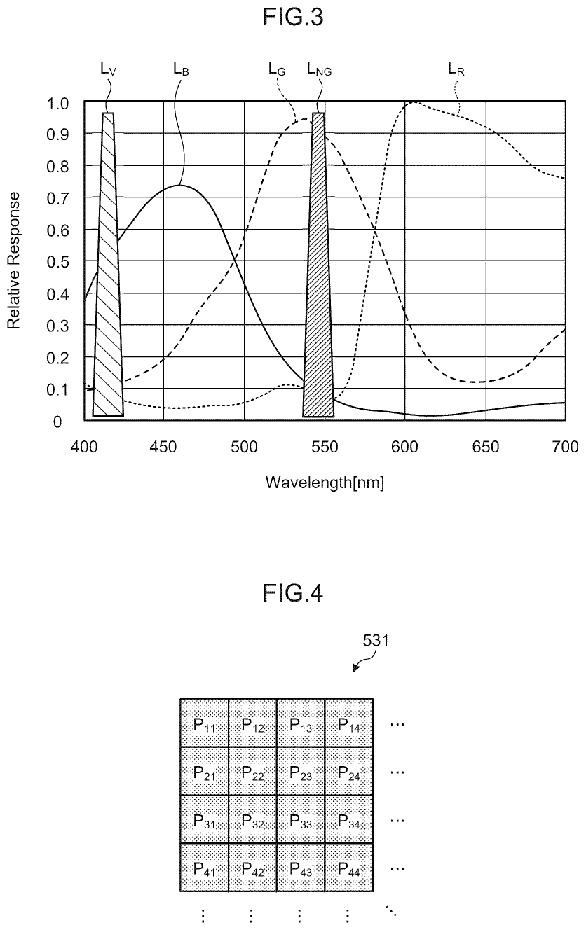

Here, a wavelength characteristic of the light emitted by each of the second light source unit 32 and the third light source unit 33 will be described. FIG. 3 is a diagram schematically illustrating the wavelength characteristic of the light emitted by each of the second light source unit 32 and the third light source unit 33. In FIG. 3, a horizontal axis represents a wavelength (nm), and a vertical axis represents the wavelength characteristic. In FIG. 3, a polygonal line L_{NG} indicates the wavelength characteristic of the first narrowband light emitted by the second light source unit 32, and a polygonal line L_{V} indicates the wavelength characteristic of the second narrowband light (excitation light) emitted by the third light source unit 33. In FIG. 3, a curve L_{B} indicates a blue wavelength band, a curve L_{G} indicates a green wavelength band, and a curve L_{R} indicates a red wavelength band.

As indicated by the polygonal line L_{NG} in FIG. 3, the second light source unit 32 emits the narrowband light having the central wavelength (peak wavelength) of 540 nm and the wavelength band of 530 nm to 550 nm. In addition, the third light source unit 33 emits the excitation light having the central wavelength (peak wavelength) of 415 nm and the wavelength band of 400 nm to 430 nm.

As described above, each of the second light source unit 32 and the third light source unit 33 emits the first narrowband light and the second narrowband light (excitation light) with different wavelength bands.

In addition, the first narrowband light is formed as light for layer discrimination in the biological tissue. Specifically, in the first narrowband light, a difference between an absorbance of a mucosal layer that is the subject and an absorbance of a muscle layer that is the subject is large enough to identify the two subjects. Therefore, in a second image for layer discrimination acquired by irradiation with the first narrowband light for layer discrimination, a region where the imaged mucosal layer appears has a smaller pixel value (luminance value) and is darker than a region where the imaged muscle layer appears. That is, in the first embodiment, it is possible to set a display mode in which the mucosal layer and the muscle layer can be easily identified by using the second image for layer discrimination for generation of a display image.

In addition, the second narrowband light (excitation light) is light for layer discrimination in the biological tissue and is different from the first narrowband light. Specifically, in the second narrowband light, a difference between the absorbance of the muscle layer that is the subject and an absorbance of a fat layer that is the subject is large enough to identify the two subjects. Therefore, in the second light image for layer discrimination acquired by irradiation with the second narrowband light for layer discrimination, a region where the imaged muscle layer appears has a smaller pixel value (luminance value) and is darker than a region where the imaged fat layer appears. That is, it is possible to set a mode in which the muscle layer and the fat layer are easily identified by using the second image for layer discrimination for generation of the display image.

Both the mucosal layer (biological mucosa) and the muscle layer are the subjects containing a large amount of myoglobin. However, a concentration of myoglobin contained is relatively high in the mucosal layer and relatively low in the muscle layer. A difference in light absorption characteristic between the mucosal layer and the muscle layer is caused by a difference in concentration of myoglobin contained in each of the mucosal layer (biological mucosa) and the muscle layer. The difference in absorbance between the mucosal layer and the muscle layer is maximum in the vicinity of a wavelength at which the absorbance of the biological mucosa has a maximum value. That is, the first narrowband light for layer discrimination is light with which a difference between the mucosal layer and the muscle layer appears larger than light having a peak wavelength in another wavelength band.

In addition, since fat has a lower absorbance for the second narrowband light for layer discrimination than the muscle layer, the pixel value (luminance value) of the region where the imaged muscle layer appears is smaller than the pixel value (luminance value) of the region where the imaged fat layer appears in the second image captured by irradiation with the second narrowband light for layer discrimination. In particular, since the second narrowband light for layer discrimination is light corresponding to a wavelength at which the absorbance of the muscle layer has a maximum value, the second narrowband light is light with which a difference between the muscle layer and the fat layer is large. That is, a difference between the pixel value (luminance value) of a muscle layer region and the pixel value (luminance value) of a fat layer region in the second image for layer discrimination is increased to an identifiable extent.

As described above, the light source device 3 irradiates the biological tissue with each of the first narrowband light and the second narrowband light. As a result, the endoscope camera head 5 described below can obtain an image in which each of the mucosal layer, the muscle layer, and the fat layer included in the biological tissue can be identified by imaging the return light from the biological tissue. In the following description, light obtained by combining the first narrowband light and the second narrowband light is expressed as special light.

In the first embodiment, the second narrowband light (excitation light) excites the advanced glycation end products generated by performing the heat treatment on the biological tissue by the energy device or the like. In a case where an amino acid and a reducing sugar are heated, a saccharification reaction (Maillard reaction) occurs. The end products resulting from the Maillard reaction are generally called the advanced glycation end products (AGEs). As a characteristic of the AGEs, it is known that a substance having a fluorescence characteristic is contained. That is, in a case where the biological tissue is subjected to the heat treatment by the energy device, the AGEs are generated when the Maillard reaction occurs by heating the amino acid and the reducing sugar in the biological tissue. The AGEs generated by the heating can visualize a state of the heat treatment by fluorescence observation. Furthermore, the AGEs are known to emit stronger fluorescence than an autofluorescent substance originally present in the biological tissue. That is, in the first embodiment, the thermally denatured region obtained by the heat treatment is visualized using the fluorescence characteristic of the AGEs generated in the biological tissue by the heat treatment using the energy device or the like. Therefore, in the first embodiment, the biological tissue is irradiated with the excitation light of blue light having a wavelength of about 415 nm for exciting the AGEs from the second light source unit 32 (excitation light). As a result, in the first embodiment, a fluorescence image (thermal denaturation image) can be observed based on the imaging signal obtained by imaging the fluorescence (for example, green light having a wavelength of 490 to 625 nm) emitted from the thermally denatured region generated from the AGEs. Therefore, in the following description, in a case where the second narrowband light is used alone, the second narrowband light is expressed as the excitation light.

### Configuration of Endoscope Camera Head

Returning to FIG. 2, the description of the configuration of the endoscope system 1 will be continued.

Next, a configuration of the endoscope camera head 5 will be described. The endoscope camera head 5 includes an optical system 51, a drive unit 52, an imaging element 53, a cut filter 54, an A/D converter 55, a P/S converter 56, an imaging recording unit 57, and an imaging control unit 58.

The optical system 51 forms the subject image condensed by the optical system 22 of the insertion unit 2 on a light receiving surface of the imaging element 53. The optical system 51 can change a focal length and a focal position. The optical system 51 is implemented using a plurality of lenses 511. The optical system 51 changes the focal length and the focal position by moving each of the plurality of lenses 511 on an optical axis L1 by the drive unit 52.

The drive unit 52 moves the plurality of lenses 511 of the optical system 51 along the optical axis L1 under the control of the imaging control unit 58. The drive unit 52 is implemented using a motor such as a stepping motor, a DC motor, or a voice coil motor, and a transmission mechanism such as a gear that transmits rotation of the motor to the optical system 51.

The imaging element 53 is implemented by using a charge coupled device (CCD) or complementary metal oxide semiconductor (CMOS) image sensor including a plurality of pixels arranged in a two-dimensional matrix. The imaging element 53 receives the subject image (light beam) formed by the optical system 51 and passing through the cut filter 54, performs the photoelectric conversion to generate the imaging signal (RAW data), and outputs the imaging signal to the A/D converter 55 under the control of the imaging control unit 58. The imaging element 53 includes a pixel unit 531 and a color filter 532.

FIG. 4 is a diagram schematically illustrating a configuration of the pixel unit 531. As illustrated in FIG. 4, in the pixel unit 531, a plurality of pixels Pₙₘ (n=an integer of 1 or more, and m=an integer of 1 or more) such as photodiodes that accumulate charges according to a light quantity are arranged in a two-dimensional matrix. The pixel unit 531 reads an image signal as the image data from a pixel Pₙₘ in a reading region arbitrarily set as a reading target among the plurality of pixels Pₙₘ, and outputs the image signal to the A/D converter 55 under the control of the imaging control unit 58.

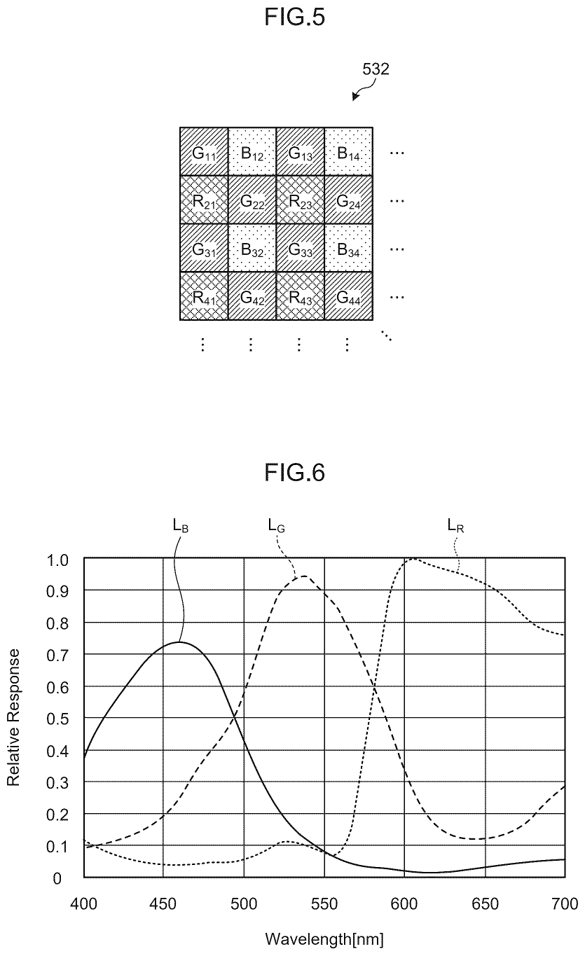

FIG. 5 is a diagram schematically illustrating a configuration of the color filter 532. As illustrated in FIG. 5, the color filter 532 is implemented by a Bayer array having 2×2 as one unit. The color filter 532 is implemented using a filter R that transmits light in the red wavelength band, two filters G that transmit light in the green wavelength band, and a filter B that transmits light in the blue wavelength band.

FIG. 6 is a diagram schematically illustrating sensitivity and a wavelength band of each filter. In FIG. 6, a horizontal axis represents the wavelength (nm), and a vertical axis represents a transmission characteristic (sensitivity characteristic). In FIG. 6, the curve L_{B} indicates the transmission characteristic of the filter B, the curve L_{G} indicates the transmission characteristic of the filter G, and the curve L_{R} indicates the transmission characteristic of the filter R.

As indicated by the curve L_{B} in FIG. 6, the filter B transmits light in the blue wavelength band. As indicated by the curve L_{G} in FIG. 6, the filter G transmits light in the green wavelength band. Further, as indicated by the curve L_{R} in FIG. 6, the filter R transmits light in the red wavelength band. In the following description, a pixel Pₙₘ in which the filter R is disposed on a light receiving surface will be described as an R pixel, a pixel Pₙₘ in which the filter G is disposed on a light receiving surface will be described as a G pixel, and a pixel Pₙₘ in which the filter B is disposed on a light receiving surface will be described as a B pixel.

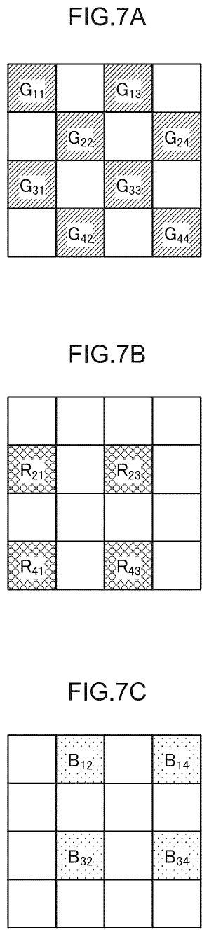

With the imaging element 53 configured as described above, in a case where the subject image formed by the optical system 51 is received, as illustrated in FIGS. 7A to 7C, color signals (an R signal, a G signal, and a B signal) of the R pixel, the G pixel, and the B pixel are generated.

Returning to FIG. 2, the description of the configuration of the endoscope system 1 will be continued.

The cut filter 54 is disposed on the optical axis L1 between the optical system 51 and the imaging element 53. The cut filter 54 is provided on a light receiving surface side (incident surface side) of the G pixel provided with the filter G that transmits at least the green wavelength band of the color filter 532. The cut filter 54 blocks light in a shorter wavelength band including the wavelength band of the excitation light, and transmits a longer wavelength band beyond the wavelength band of the excitation light.

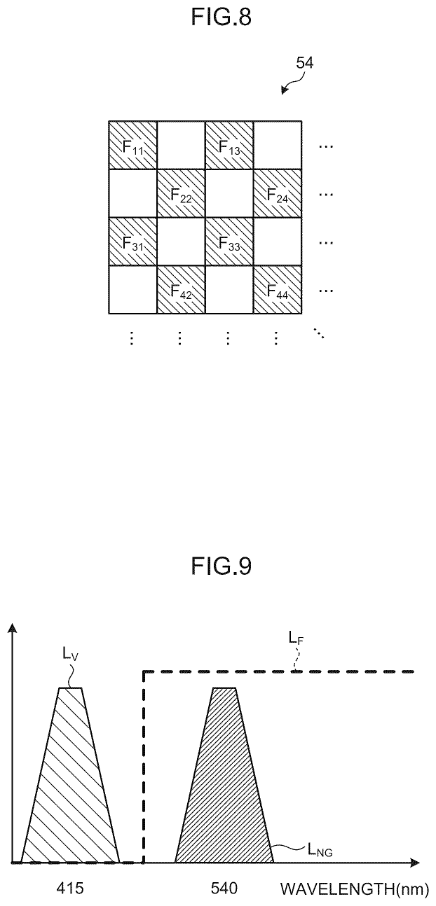

FIG. 8 is a diagram schematically illustrating a configuration of the cut filter 54. As illustrated in FIG. 8, a filter F₁₁ included in the cut filter 54 is disposed at a position where a filter G₁₁ (see FIG. 5) is disposed, on the light receiving surface side immediately above the filter G₁₁.

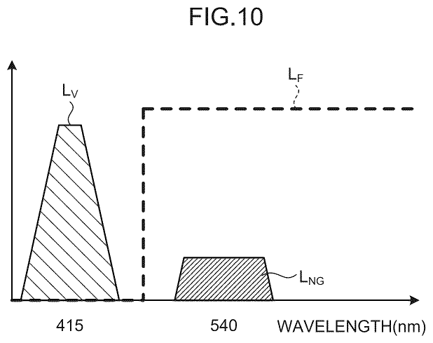

FIGS. 9 and 10 are diagrams schematically illustrating a transmission characteristic of the cut filter 54. In FIGS. 9 and 10, a horizontal axis represents the wavelength (nm), and a vertical axis represents the transmission characteristic. In FIG. 9, a polygonal line L_{F} indicates the transmission characteristic of the cut filter 54, the polygonal line L_{NG} indicates the wavelength characteristic of the first narrowband light, and the polygonal line L_{V} indicates the wavelength characteristic of the second narrowband light (excitation light). FIG. 10 illustrates the transmission characteristics when the fluorescence from the thermally denatured region is received in a case where the thermally denatured region is irradiated with the excitation light.

As illustrated in FIG. 9, the cut filter 54 blocks the wavelength band of the second narrowband light (excitation light) and transmits the longer wavelength band beyond the wavelength band of the second narrowband light (excitation light). Specifically, the cut filter 54 blocks the light in the shorter wavelength band of 400 nm to less than 430 nm including the wavelength band of the second narrowband light (excitation light), and transmits the light in the longer wavelength band beyond the wavelength band of 400 nm to 430 nm including the second narrowband light (excitation light). Further, as illustrated in FIG. 10, when the thermally denatured region is irradiated with the excitation light, the cut filter 54 transmits the fluorescence in the wavelength band from the thermally denatured region.

Returning to FIG. 2, the description of the configuration of the endoscope camera head 5 will be continued.

The A/D converter 55 executes A/D conversion processing on the analog imaging signal input from the imaging element 53, and outputs the analog imaging signal to the P/S converter 56 under the control of the imaging control unit 58. The A/D converter 55 is implemented by using an A/D conversion circuit or the like.

The P/S converter 56 performs parallel/serial conversion on the digital imaging signal input from the A/D converter 55, and outputs the imaging signal subjected to the parallel/serial conversion to the control device 9 via the first transmission cable 6 under the control of the imaging control unit 58. The P/S converter 56 is implemented by using a P/S conversion circuit or the like. In the first embodiment, an E/O converter that converts the imaging signal into an optical signal may be provided instead of the P/S converter 56, and the imaging signal may be output to the control device 9 by the optical signal, or the imaging signal may be transmitted to the control device 9 by wireless communication such as Wireless Fidelity (Wi-Fi) (registered trademark).

The imaging recording unit 57 records various types of information (for example, pixel information of the imaging element 53 and the characteristic of the cut filter 54) regarding the endoscope camera head 5. Furthermore, the imaging recording unit 57 records various types of setting data and control parameters transmitted from the control device 9 via the first transmission cable 6. The imaging recording unit 57 is implemented using a nonvolatile memory or a volatile memory.

The imaging control unit 58 controls an operation of each of the drive unit 52, the imaging element 53, the A/D converter 55, and the P/S converter 56 based on the setting data received from the control device 9 via the first transmission cable 6. The imaging control unit 58 is implemented by using a timing generator (TG), a processor including hardware such as an application specific integrated circuit (ASIC) or a CPU, and a memory that is a temporary storage area used by the processor.

### Configuration of Control Device

Next, a configuration of the control device 9 will be described.

The control device 9 includes an S/P converter 91, an image processor 92, an input unit 93, a recording unit 94, and a control unit 95.

The S/P converter 91 performs serial/parallel conversion on the image data received from the endoscope camera head 5 via the first transmission cable 6 and outputs the image data to the image processor 92 under the control of the control unit 95. In a case where the endoscope camera head 5 outputs the imaging signal as the optical signal, an O/E converter that converts the optical signal into an electric signal may be provided instead of the S/P converter 91. Furthermore, in a case where the endoscope camera head 5 transmits the imaging signal by wireless communication, a communication module capable of receiving a wireless signal may be provided instead of the S/P converter 91.

The image processor 92 executes predetermined image processing on the imaging signal of the parallel data input from the S/P converter 91 and outputs the imaging signal to the display device 7 under the control of the control unit 95. Here, the predetermined image processing is demosaic processing, white balance processing, gain adjustment processing, y correction processing, format conversion processing, or the like. The image processor 92 is implemented by using a processor that is a processing device including hardware such as a GPU or an FPGA and a memory that is a temporary storage area used by the processor.

Furthermore, in a case where the light source device 3 performs irradiation with the special light, the image processor 92 executes the image processing on signal values of the G pixel and the B pixel included in the imaging signal input from the endoscope camera head 5 via the S/P converter 91 to generate a pseudo color image (narrowband image). In this case, the signal value of the G pixel includes deep mucosal layer information of the subject. Furthermore, the signal value of the B pixel includes surface mucosal layer information of the subject. Therefore, the image processor 92 executes the image processing such as gain control processing, pixel interpolation processing, and mucosal enhancement processing on the signal value of each of the G pixel and the B pixel included in the imaging signal to generate the pseudo color image, and outputs the pseudo color image to the display device 7. Here, the pseudo color image is an image generated using only the signal value of the G pixel and the signal value of the B pixel. The image processor 92 acquires a signal value of the R pixel, but does not use the signal value for generating the pseudo color image and deletes the signal value.

Furthermore, in a case where the light source device 3 performs irradiation with the excitation light, the image processor 92 executes the image processing on the signal value of each of the G pixel and the B pixel included in the imaging signal input from the endoscope camera head 5 via the S/P converter 91 to generate the fluorescence image (pseudo color image). In this case, the signal value of the G pixel includes fluorescence information emitted from a heat treatment region. Furthermore, the B pixel includes background information regarding a biological tissue around the heat treatment region. Therefore, the image processor 92 executes the image processing such as the gain control processing, the pixel interpolation processing, and the mucosal enhancement processing on the signal value of each of the G pixel and the B pixel included in the image data to generate the fluorescence image (pseudo color image), and outputs the fluorescence image (pseudo color image) to the display device 7. In this case, the image processor 92 executes the gain control processing of making a gain for the signal value of the G pixel larger than a gain for the signal value of the G pixel at the time of normal light observation, and making a gain for the signal value of the B pixel smaller than a gain for the signal value of the B pixel at the time of the normal light observation. Furthermore, the image processor 92 executes the gain control processing such that the signal value of the G pixel and the signal value of the B pixel are the same as each other (1:1).

The input unit 93 receives various operations related to the endoscope system 1 and outputs the received operations to the control unit 95. The input unit 93 is implemented using a mouse, a foot switch, a keyboard, a button, a switch, a touch panel, or the like.

The recording unit 94 is implemented by using a recording medium such as a volatile memory, a nonvolatile memory, a solid state drive (SSD), a hard disk drive (HDD), or a memory card. The recording unit 94 records data including various parameters and the like necessary for an operation of the endoscope system 1. Furthermore, the recording unit 94 includes a program recording unit 941 that records various programs for operating the endoscope system 1.

The control unit 95 is implemented by using a processor including hardware such as an FPGA or a CPU, and a memory that is a temporary storage area used by the processor. The control unit 95 integrally controls each unit included in the endoscope system 1. Specifically, the control unit 95 reads and executes the program recorded in the program recording unit 941 in a work area of the memory, and controls each component and the like through the execution of the program by the processor, so that the hardware and software cooperate with each other to implement a functional module matching a predetermined purpose. Specifically, the control unit 95 includes an acquisition unit 951, a generation unit 952, a setting unit 953, a specification unit 954, an alignment unit 955, a determination unit 956, an output control unit 957, and a learning unit 958.

The acquisition unit 951 acquires the imaging signal of the white light generated by the endoscope camera head 5 when the light source device 3 irradiates the biological tissue with the white light via the S/P converter 91 and the image processor 92. In addition, the acquisition unit 951 acquires the setting information regarding setting of the region of interest for the biological tissue and thermal denaturation information regarding the thermally denatured region in which thermal denaturation has occurred by the heat treatment for the biological tissue via the S/P converter 91 and the image processor 92. Specifically, the acquisition unit 951 acquires a first image as the setting information, and acquires a second image as the thermal denaturation information. Here, the first image is a special light image. The second image is the fluorescence image.

The generation unit 952 generates the first image based on a first imaging signal acquired by the acquisition unit 951. Here, the first image is the special light image. The first image is the setting information in the first embodiment. In addition, the generation unit 952 generates the second image based on a second imaging signal acquired by the acquisition unit 951. Here, the second image is the fluorescence image. Furthermore, the generation unit 952 generates a white light image based on a white light image signal acquired by the acquisition unit 951.

The setting unit 953 sets the region of interest based on feature data included in the first image generated by the generation unit 952. Specifically, the setting unit 953 determines whether or not the luminance value is a predetermined value or more for each pixel forming the first image generated by the generation unit 952, and sets, as the region of interest, a region formed by a plurality of pixels whose luminance values are the predetermined value or more.

The specification unit 954 specifies the thermally denatured region based on the second image generated by the generation unit 952. Specifically, the specification unit 954 determines whether or not the luminance value is the predetermined value or more for each pixel forming the second image, and specifies, as the thermally denatured region, a region formed by a plurality of pixels whose luminance values are the predetermined value or more.

The alignment unit 955 executes alignment processing of the first image and the second image. For example, the alignment unit 955 executes the alignment processing of the first image and the second image based on a position where the feature data of each pixel forming the first image is aligned with the feature data of each pixel forming the second image. Here, the feature data is, for example, the pixel value, the luminance value, an edge, or a contrast.

The determination unit 956 determines whether or not there is a thermally denatured region outside the region of interest based on the setting information and the thermal denaturation information. Specifically, the determination unit 956 determines whether or not there is a thermally denatured region outside the region of interest based on the setting information of the first image and the thermal denaturation information of the second image, the first image and the second image being subjected to the alignment processing by the alignment unit 955.

In a case where the determination unit 956 determines that there is a thermally denatured region outside the region of interest, the output control unit 957 outputs, to the display device 7, support information indicating that there is a thermally denatured region outside the region of interest. Specifically, the output control unit 957 outputs, as the support information, one or more of a message, a figure, and a sound indicating that there is a thermally denatured region outside the region of interest to the display device 7 to cause the display device 7 to display the support information. The output control unit 957 may generate the display image by identifiably superimposing the thermally denatured region generated outside the region of interest and specified by the specification unit 954 and the thermally denatured region in the region of interest on the white light image generated by the generation unit 952, and output, as the support information, the display image to the display device 7. In addition, the output control unit 957 may generate the display image in which the thermally denatured region generated outside the region of interest and specified by the specification unit 954 and the thermally denatured region in the region of interest can be identified, and output, as the support information, the display image to the display device 7.

The learning unit 958 generates a trained model by performing machine learning using training data in which the fluorescence image generated based on the imaging signal generated by imaging the light emitted from the thermally denatured region by irradiating the biological tissue with the excitation light and the white light image generated based on the imaging signal generated by imaging the return light by irradiating the biological tissue with the white light are input data, and the support information indicating that there is a thermally denatured region included in the fluorescence image outside the region of interest included in the white light image is output data. Specifically, the learning unit 958 may generate the trained model by performing machine learning using the training data in which the fluorescence image obtained by imaging the fluorescence by irradiating the biological tissue with the excitation light and the special light image (narrowband light observation image) or the white light image obtained by performing imaging by irradiating the biological tissue with the narrowband light having a wavelength determined according to an absorption rate of hemoglobin are the input data, and the support information indicating that there is a thermally denatured region included in the fluorescence image outside the region of interest included in the white light image is the output data. Here, the trained model includes a neural network in which each layer includes one or more nodes.

Furthermore, a type of the machine learning is not particularly limited, and for example, it is sufficient if the training data and learning data in which the fluorescence image and the special light image (narrowband light observation image) or the white light image are associated with annotation information that designates a position or a region of the thermally denatured region positioned outside the region of interest based on the fluorescence image and the special light image (narrowband light observation image) or the white light image are prepared, and the training data and the learning data are input to a calculation model based on a multilayer neural network to perform learning.

Furthermore, as a method for the machine learning, for example, a method based on a deep neural network (DNN) of the multilayer neural network such as a convolutional neural network (CNN) or a 3D-CNN is used. Furthermore, as the method for the machine learning, a method based on a recurrent neural network (RNN), a long short-term memory unit (LSTM) obtained by extending the RNN, or the like may be used. A control unit of a learning device different from the control device 9 may execute such functions to generate the trained model. It is a matter of course that the function of the learning unit 958 may be provided in the image processor 92.

### Processing in Control Device

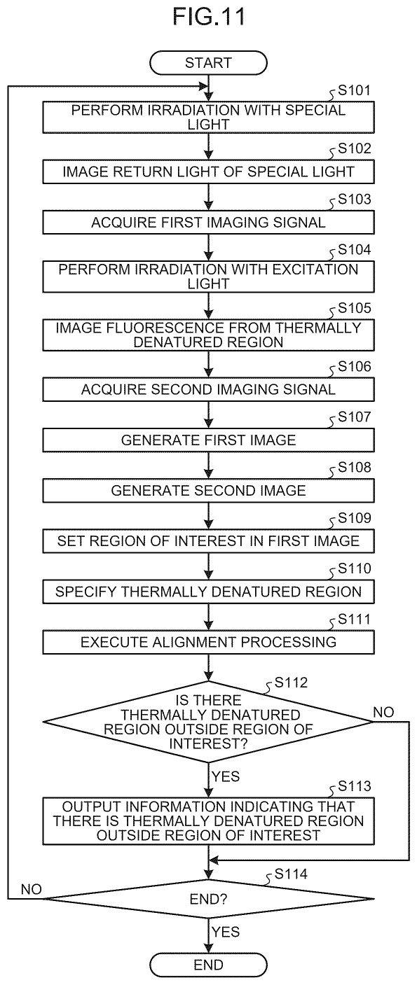

Next, processing executed by the control device 9 will be described. FIG. 11 is a flowchart illustrating an outline of the processing executed by the control device 9.

As illustrated in FIG. 11, first, the control unit 95 controls the light source control unit 34 of the light source device 3 to cause the second light source unit 32 and the third light source unit 33 to emit light and supply the special light to the insertion unit 2, thereby irradiating the biological tissue with the special light (step S101).

Subsequently, the control unit 95 controls the imaging control unit 58 to cause the imaging element 53 to image the return light of the special light from the biological tissue (step S102).

Thereafter, the acquisition unit 951 acquires the first imaging signal generated by imaging performed by the imaging element 53 of the endoscope camera head 5 (step S103).

Subsequently, the control unit 95 controls the light source control unit 34 of the light source device 3 to cause the third light source unit 33 to emit light to perform irradiation with the excitation light (step S104).

Thereafter, the control unit 95 controls the imaging control unit 58 to cause the imaging element 53 to image the fluorescence from the thermally denatured region of the biological tissue (step S105).

Subsequently, the acquisition unit 951 acquires the second imaging signal generated by imaging performed by the imaging element 53 of the endoscope camera head 5 (step S106).

Thereafter, the generation unit 952 generates the first image based on the first imaging signal acquired by the acquisition unit 951 (step S107). Here, the first image is the special light image. The first image is the setting information in the first embodiment.

Subsequently, the generation unit 952 generates the second image based on the second imaging signal acquired by the acquisition unit 951 (step S108). Here, the second image is the fluorescence image.

Thereafter, the setting unit 953 sets the region of interest based on the feature data included in the first image (step S109).

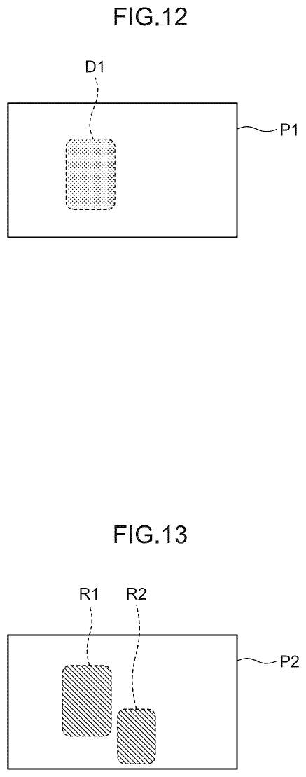

FIG. 12 is a diagram schematically illustrating the region of interest set by the setting unit 953 for the first image. As illustrated in FIG. 12, the setting unit 953 sets the region of interest based on the feature data included in the first image. Specifically, the setting unit 953 determines whether or not the luminance value is the predetermined value or more for each pixel forming a first image P1, and sets, as a region D1 of interest, a region formed by a plurality of pixels whose luminance values are the predetermined value or more.

After step S109, the specification unit 954 specifies the thermally denatured region based on the second image (step S110).

FIG. 13 is a diagram schematically illustrating the thermally denatured region specified by the specification unit 954 for the second image. As illustrated in FIG. 13, the specification unit 954 specifies the thermally denatured region based on the second image. Specifically, the specification unit 954 determines whether or not the luminance value is the predetermined value or more for each pixel forming a second image P2, and specifies, as thermally denatured regions R1 and R2, regions formed by a plurality of pixels whose luminance values are the predetermined value or more. The specification unit 954 may specify the thermally denatured region (fluorescent region) generated by the heat treatment by comparing a reference image based on the imaging signal generated by imaging performed by the imaging element 53 of the endoscope camera head 5 before the heat treatment by a resection treatment tool or the like with the fluorescent image that is the second image. In this case, the reference image may be recorded in advance in the recording unit 94.

After step S110, the alignment unit 955 executes the alignment processing of the first image and the second image (step S111).

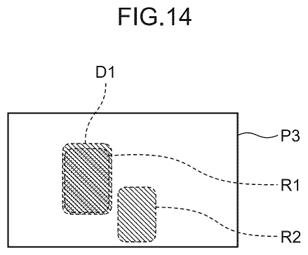

FIG. 14 is a diagram schematically illustrating the alignment processing executed by the alignment unit 955. As illustrated in FIG. 14, the alignment unit 955 executes the alignment processing by using a known technology such that a position of the feature data included in the first image P1 and a position of the feature data included in the second image P2 are aligned with each other. For example, the alignment unit 955 executes the alignment processing of the first image P1 and the second image P2 based on a position where the feature data of each pixel forming the first image P1 is aligned with the feature data of each pixel forming the second image P2. Here, the feature data includes, for example, the pixel value, the luminance value, the edge, or the contrast.

After step S111, the determination unit 956 determines whether or not there is a thermally denatured region outside the region of interest based on the setting information of the first image and the thermal denaturation information of the second image, the first image and the second image being subjected to the alignment processing by the alignment unit 955 (step S112). In FIG. 14, the determination unit 956 determines that there is a thermally denatured region R2 outside the region D1 of interest based on the region D1 of interest of the first image P1 subjected to the alignment processing executed by the alignment unit 955 and the thermally denatured regions R1 and R2 of the second image P2. In FIG. 14, the determination unit 956 determines that the thermally denatured region R2 is outside the region D1 of interest. In a case where the determination unit 956 determines that there is a thermally denatured region outside the region of interest (step S112: Yes), the control device 9 proceeds to step S113 described below. On the other hand, in a case where the determination unit 956 determines that there is no thermally denatured region outside the region of interest (step S112: No), the control device 9 proceeds to step S114 described below.

In step S113, the output control unit 957 outputs, to the display device 7, the support information indicating that there is a thermally denatured region outside the region of interest. Specifically, the output control unit 957 generates the display image in which information indicating that there is a thermally denatured region R2 outside the region D1 of interest is superimposed on the first image P1, and outputs the display image to the display device 7 as the support information to display the display image. In this case, the output control unit 957 may output, to the display device 7, the display image (for example, see FIG. 14) in which the thermally denatured region R1 inside the region D1 of interest and the thermally denatured region R2 outside the region D1 of interest are identifiably superimposed on the first image P1, and at least one of a message, a figure, and a symbol indicating that there is a thermally denatured region outside the region of interest as the support information to display the display image. It is a matter of course that the output control unit 957 may generate the display image in which one or more of the message and the figure indicating that there is a thermally denatured region outside the region of interest are superimposed on the first image, and output the display image to the display device 7 to display the display image. As a result, a user can grasp that there is a thermally denatured region outside the region of interest. The output control unit 957 may simply output the message, the figure, or the like indicating that there is a thermally denatured region outside the region of interest to the display device 7 to cause the display device 7 to display the message, the figure, or the like.

Subsequently, the control unit 95 determines whether or not an end signal for ending the observation of the subject by the endoscope system 1 has been input from the input unit 93 (step S114). In a case where the control unit 95 determines that the end signal for ending the observation of the subject by the endoscope system 1 has been input from the input unit 93 (step S114: Yes), the control device 9 ends the processing. On the other hand, in a case where the control unit 95 determines that the end signal for ending the observation of the subject by the endoscope system 1 has not been input from the input unit 93 (step S114: No), the control device 9 returns to step S101 described above.

According to the first embodiment described above, in a case where the determination unit 956 determines that there is a thermally denatured region outside the region of interest, the output control unit 957 outputs information indicating that there is a thermally denatured region outside the region of interest to the display device 7, so that it is possible to grasp the presence or absence of a thermally denatured region outside the region of interest.

Furthermore, according to the first embodiment, the setting unit 953 sets the region of interest based on the feature data included in the first image, so that it is possible to assist the user in performing a medical procedure.

Further, according to the first embodiment, the specification unit 954 specifies the thermally denatured region based on the second image, so that it is possible to easily specify the thermally denatured region.

Furthermore, in the first embodiment, the learning unit 958 is provided in the control device 9, but the present disclosure is not limited thereto, and the learning unit 958 that generates the trained model may be provided in a device different from the control device 9, such as a learning device or a server connectable via a network.

Furthermore, in the first embodiment, the output control unit 957 may generate the display image by identifiably superimposing, on the white light image generated by the generation unit 952, the thermally denatured region generated outside the region of interest specified by the specification unit 954 and the thermally denatured region in the region of interest, and output the display image to the display device 7 as the support information. As a result, the user can grasp the thermally denatured region outside the region of interest on the white light image.

In addition, in the first embodiment, the output control unit 957 may generate the display image in which the thermally denatured region generated outside the region of interest specified by the specification unit 954 and the thermally denatured region in the region of interest can be identified, and output the display image to the display device 7 as the support information. As a result, the user can easily identify the thermally denatured region generated outside the region of interest and the thermally denatured region in the region of interest.

### Second Embodiment

Next, a second embodiment will be described. An endoscope system according to the second embodiment has the same configuration as the endoscope system 1 according to the first embodiment described above, and processing executed by a control device 9 is different. Specifically, in the first embodiment described above, the region of interest is set according to the feature data of the special light image serving as the first image, but in the second embodiment, a first image is a white light image, and a region of interest is set according to an instruction signal input from an input unit 93. Therefore, processing executed by the control device 9 included in an endoscope system 1 according to the second embodiment will be described below.

### Processing in Control Device

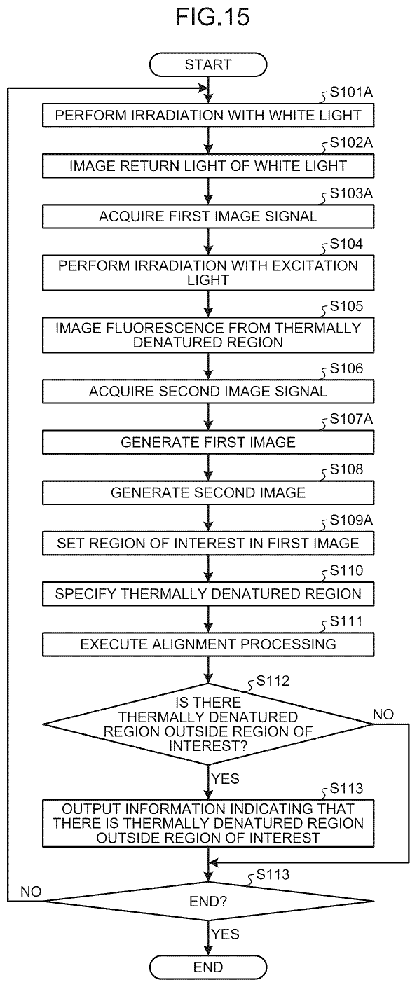

FIG. 15 is a flowchart illustrating an outline of the processing executed by the control device 9 according to the second embodiment. In FIG. 15, the control device 9 executes step S101A, step S102A, step S107A, and step S109A instead of step S101, step S102, step S107, and step S109 described above in FIG. 11, and executes processing similar to the processing in FIG. 11 described above for other steps. Therefore, in FIG. 15, step S101A, step S102A, step S107A, and step S109A will be described.

As illustrated in FIG. 15, first, a control unit 95 controls a light source control unit 34 of a light source device 3 to cause a first light source unit 31 to emit light and supply white light to an insertion unit 2, thereby irradiating a biological tissue with the white light (step S101A).

Subsequently, the control unit 95 controls an imaging control unit 58 to cause an imaging element 53 to image return light of the white light from the biological tissue (step S102A).

Thereafter, an acquisition unit 951 acquires a first imaging signal generated by imaging performed by the imaging element 53 of an endoscope camera head 5 (step S103A). In this case, the acquisition unit 951 acquires, as setting information, instruction information for instructing an annotation input by a user via the input unit 93 for the white light image displayed on a display device 7. After step S103A, the control device 9 proceeds to step S104.

In step S107A, the generation unit 952 generates the first image based on the first imaging signal acquired by the acquisition unit 951. Here, the first image is the white light image. After step S107A, the control device 9 proceeds to step S108.

In step S109A, the setting unit 953 sets the region of interest in the white light image displayed on the display device 7 according to the instruction signal for instructing the annotation by the user via the input unit 93, the instruction signal being acquired by the acquisition unit 951. For example, the setting unit 953 sets the instruction signal corresponding to a region instructed by the user operating the input unit 93 as the region of interest as the annotation. After step S109A, the control device 9 proceeds to step S110.

According to the second embodiment described above, it is possible to obtain an effect similar to that of the first embodiment described above, that is, it is possible to grasp the presence or absence of the thermally denatured region outside the region of interest.

### Third Embodiment

Next, a third embodiment will be described. In the first embodiment described above, the setting unit 953 sets the region of interest based on the feature data included in the first image, but in the third embodiment, a medical device different from a control device detects a region of interest and sets the region of interest based on a result of the detection. Therefore, an endoscope system according to the third embodiment will be described below. Note that the same components as those of the endoscope system 1 according to the first embodiment described above are denoted by the same reference signs, and a detailed description thereof will be omitted.

### Configuration of Endoscope System

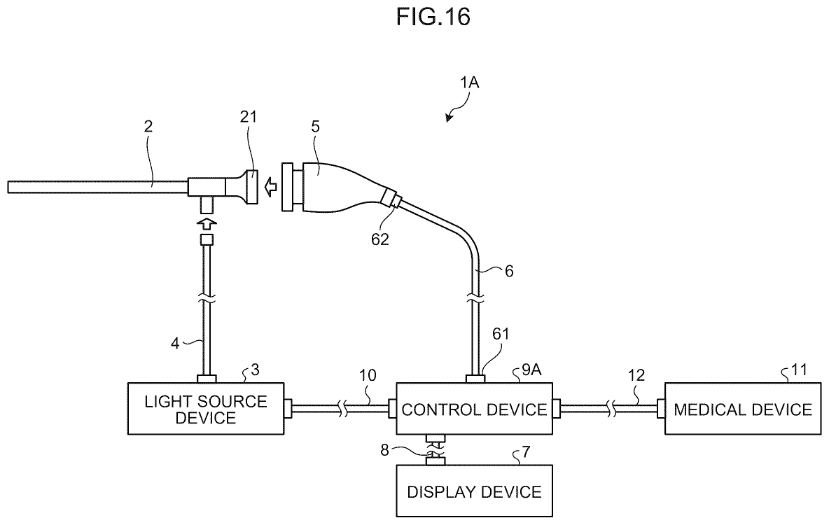

FIG. 16 is a diagram illustrating a schematic configuration of the endoscope system according to the third embodiment. An endoscope system 1A illustrated in FIG. 16 further includes a medical device 11 and a fourth transmission cable 12 in addition to the configuration of the endoscope system 1 according to the first embodiment described above.

The medical device 11 is implemented by using a processor that is a processing device including hardware such as a GPU, an FPGA, or a CPU, and a memory that is a temporary storage area used by the processor. The medical device 11 acquires various types of information from a control device 9 via the fourth transmission cable 12, and outputs the acquired various types of information to the control device 9. In addition, the medical device 11 performs machine learning using training data in which a plurality of images and annotation information added with the region of interest included in each of the plurality of images are associated with each other, and outputs, to the control device 9, position information for setting the region of interest by using a learning model that uses a white light image as input data and outputs a position of the region of interest in the white light image as output data. Here, the machine learning is deep learning or the like.

The fourth transmission cable 12 has one end detachably connected to the control device 9, and the other end detachably connected to the medical device 11. The fourth transmission cable 12 transmits various types of information from the control device 9 to the medical device 11, and transmits various types of information from the medical device 11 to the control device 9.

### Processing in Control Device

Next, processing executed by the control device 9 will be described.

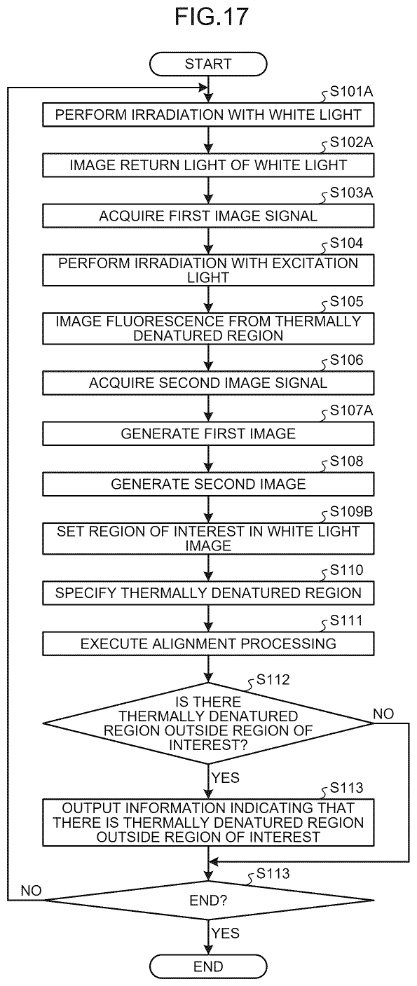

FIG. 17 is a flowchart illustrating an outline of the processing executed by the control device 9. In FIG. 17, the control device 9 executes step S109B instead of step S109A described above in FIG. 15, and executes processing similar to the processing in FIG. 15 described above for other steps. Therefore, in FIG. 17, step S109B will be described.

In step S109B, a setting unit 953 sets the region of interest in the white light image based on the position information for setting the region of interest input from the medical device 11 and acquired by an acquisition unit 951. For example, the setting unit 953 sets the region of interest of the white light image based on the position information for setting, as the region of interest, a region such as a tumor included in the white light image by using the learning model included in the medical device 11. After step S109B, the control device 9 proceeds to step S110.

According to the third embodiment described above, it is possible to obtain an effect similar to that of the first embodiment described above, that is, it is possible to grasp the presence or absence of the thermally denatured region outside the region of interest.

### Fourth Embodiment

Next, a fourth embodiment will be described. In the first embodiment described above, the control device 9 determines the presence or absence of the thermally denatured region outside the region of interest. However, in the fourth embodiment, a medical device that determines the presence or absence of a thermally denatured region outside a region of interest and outputs a result of the determination is separately provided. Hereinafter, a configuration of an endoscope system according to the fourth embodiment will be described. Note that the same components as those of the endoscope system 1 according to the first embodiment described above are denoted by the same reference signs, and a detailed description thereof will be omitted.

### Configuration of Endoscope System

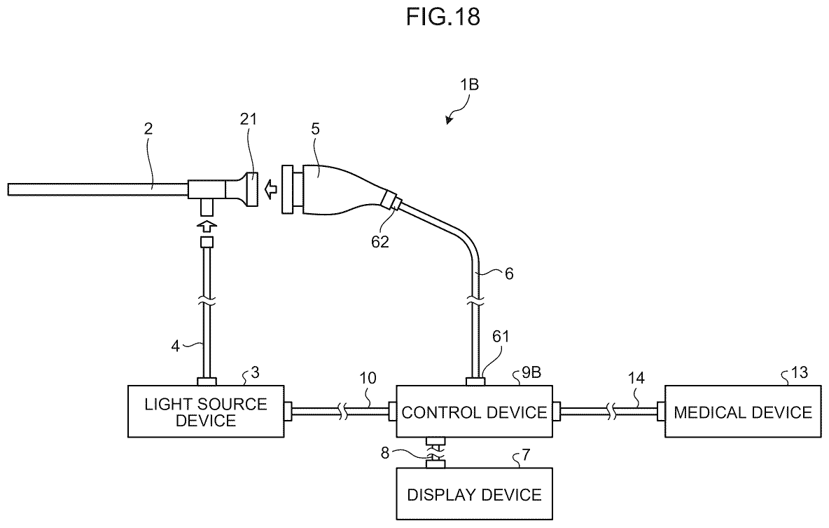

FIG. 18 is a diagram illustrating a schematic configuration of the endoscope system according to the fourth embodiment. An endoscope system 1B illustrated in FIG. 18 includes a control device 9B instead of the control device 9 according to the first embodiment described above. Furthermore, the endoscope system 1B further includes a medical device 13 and a fifth transmission cable 14 in addition to the configuration of the endoscope system 1 according to the first embodiment described above.

The control device 9B is implemented by using a processor that is a processing device including hardware such as a GPU, an FPGA, or a CPU, and a memory that is a temporary storage area used by the processor. The control device 9B integrally controls operations of a light source device 3, an endoscope camera head 5, a display device 7, and the medical device 13 via each of a first transmission cable 6, a second transmission cable 8, a third transmission cable 10, and the fifth transmission cable 14 according to a program recorded in the memory. The control device 9B is different from the control unit 95 according to the first embodiment described above in that functions of an acquisition unit 951, a generation unit 952, a setting unit 953, a specification unit 954, an alignment unit 955, a determination unit 956, an output control unit 957, and a learning unit 958 are not provided.

The medical device 13 is implemented by using a processor that is a processing device including hardware such as a GPU, an FPGA, or a CPU, and a memory that is a temporary storage area used by the processor. The medical device 13 acquires various types of information from the control device 9B via the fifth transmission cable 14, and outputs the acquired various types of information to the control device 9B. Note that a detailed functional configuration of the medical device 13 will be described below.

The fifth transmission cable 14 has one end detachably connected to the control device 9B, and the other end detachably connected to the medical device 13. The fifth transmission cable 14 transmits various types of information from the control device 9B to the medical device 13, and transmits various types of information from the medical device 13 to the control device 9B.

### Functional Configuration of Medical Device

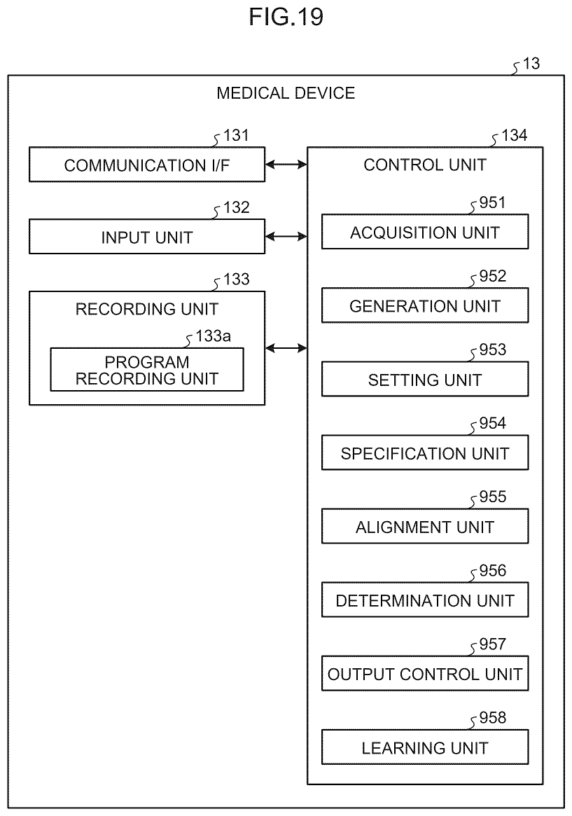

Next, a functional configuration of the medical device 13 will be described. FIG. 19 is a block diagram illustrating a functional configuration of the medical device 13. As illustrated in FIG. 19, the medical device 13 includes a communication interface (I/F) 131, an input unit 132, a recording unit 133, and a control unit 134.

The communication I/F 131 is an interface for communicating with the control device 9B via the fifth transmission cable 14. The communication I/F 131 receives various types of information from the control device 9B according to a predetermined communication standard, and outputs the received various types of information to the control unit 134.

The input unit 132 receives inputs of various operations related to the endoscope system 1B, and outputs the received operations to the control unit 134. The input unit 132 is implemented using a mouse, a foot switch, a keyboard, a button, a switch, a touch panel, and the like.

The recording unit 133 is implemented by using a recording medium such as a volatile memory, a nonvolatile memory, an SSD, an HDD, or a memory card. The recording unit 133 records data including various parameters and the like necessary for an operation of the medical device 13. Furthermore, the recording unit 133 includes a program recording unit 133a that records various programs for operating the medical device 13.

The control unit 134 is implemented by using a processor including hardware such as an FPGA or a CPU, and a memory that is a temporary storage area used by the processor. The control unit 134 integrally controls each unit included in the medical device 13. The control unit 134 has the same function as the control unit 95 according to the first embodiment described above. Specifically, the control unit 134 includes an acquisition unit 951, a generation unit 952, a setting unit 953, a specification unit 954, an alignment unit 955, a determination unit 956, an output control unit 957, and a learning unit 958.

The medical device 13 configured as described above executes processing similar to that of the control device 9 according to the first embodiment described above, and outputs a result of the processing to the control device 9B. In this case, the control device 9B outputs information corresponding to the presence or absence of the thermally denatured region outside the region of interest, which is the processing result of the medical device 13, to the display device 7.

According to the fourth embodiment described above, it is possible to obtain an effect similar to that of the first embodiment described above, that is, it is possible to grasp the presence or absence of the thermally denatured region outside the region of interest.

### Fifth Embodiment

Next, a fifth embodiment will be described. In the first embodiment described above, the output control unit 957 outputs a result of determining the presence or absence of the thermally denatured region outside the region of interest by the determination unit 956 to the display device 7, but in the fifth embodiment, a light source device projects a thermally denatured region outside a region of interest onto a biological tissue. Therefore, a functional configuration of a main part of an endoscope system according to the fifth embodiment will be described. Note that, hereinafter, the same components as those of the endoscope system 1 according to the first embodiment described above are denoted by the same reference signs, and a detailed description thereof will be omitted.

### Functional Configuration of Main Part of Endoscope System

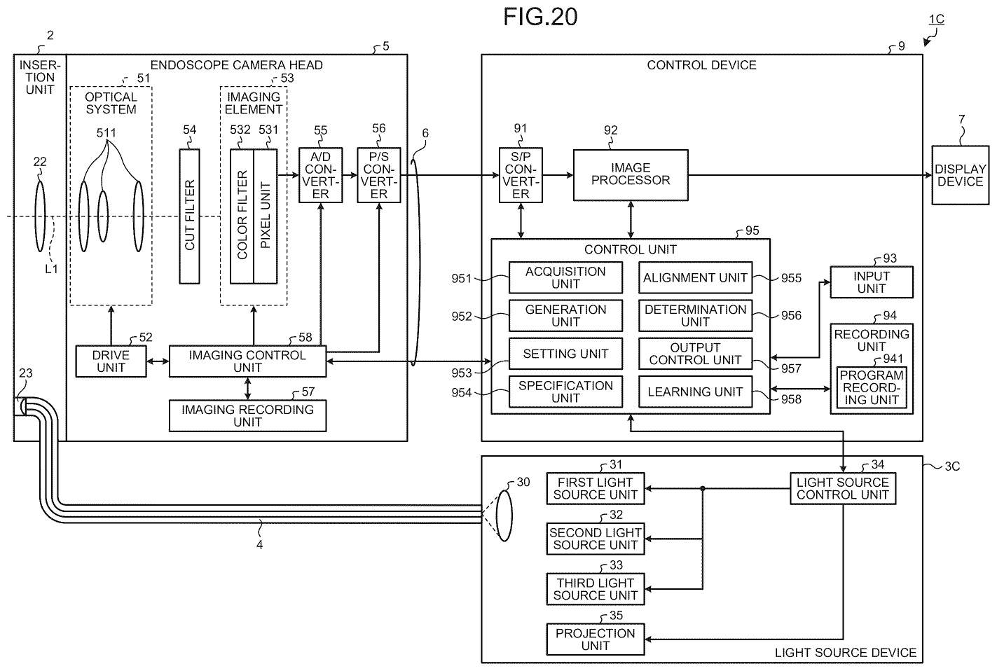

FIG. 20 is a diagram illustrating a functional configuration of a main part of an endoscope system 1C according to the fifth embodiment. The endoscope system 1C illustrated in FIG. 20 includes a light source device 3C instead of the light source device 3 of the endoscope system 1 according to the first embodiment described above.

The light source device 3C further includes a projection unit 35 that projects information regarding the region of interest and the thermally denatured region outside the region of interest toward the biological tissue in addition to the configuration of the light source device 3 according to the first embodiment described above.

The projection unit 35 projects support information including the information regarding the region of interest input from a control device 9 and the information regarding the thermally denatured region outside the region of interest onto the biological tissue via a condenser lens 30 and a light guide 4 under the control of a light source control unit 34. Specifically, the projection unit 35 identifiably projects, as the support information, the information indicating the region of interest and the information indicating the thermally denatured region outside the region of interest onto each of a region of the biological tissue that corresponds to the region of interest and a region of the biological tissue that corresponds to the thermally denatured region outside the region of interest. For example, the projection unit 35 projects each of the region of the biological tissue that corresponds to the region of interest and the region of the biological tissue that corresponds to the thermally denatured region outside the region of interest in blue and green. In the fifth embodiment, the projection unit 35 functions as a projector.

According to the fifth embodiment described above, it is possible to obtain an effect similar to that of the first embodiment described above, that is, it is possible to grasp the presence or absence of the thermally denatured region outside the region of interest.

### Other Embodiments

Various embodiments can be formed by appropriately combining a plurality of constituent elements disclosed in the endoscope systems according to the first to fifth embodiments of the present disclosure described above. For example, some constituent elements may be deleted from all the constituent elements described in the endoscope systems according to the embodiments of the present disclosure described above. Furthermore, the constituent elements described in the endoscope systems according to the embodiments of the present disclosure described above may be appropriately combined.

Furthermore, in the endoscope systems according to the first to fifth embodiments of the present disclosure, the constituent elements are connected to each other in a wired manner, or may be connected wirelessly via a network.

Furthermore, in the first to fifth embodiments of the present disclosure, the function of the control unit included in the endoscope system, and the functional modules of the acquisition unit 951, the generation unit 952, the setting unit 953, the specification unit 954, the alignment unit 955, the determination unit 956, and the output control unit 957 may be provided in a server or the like connectable via a network. It is a matter of course that a server may be provided for each functional module.

In addition, in the first to fifth embodiments of the present disclosure, an example in which the endoscope system is used for the transurethral resection of the bladder tumor has been described, but the present disclosure is not limited thereto, and the endoscope system can be applied to various medical procedures for resection of a lesion by, for example, the energy device or the like.

Furthermore, in the endoscope systems according to the first to fifth embodiments of the present disclosure, the “unit” described above can be replaced with “means”, “circuit”, or the like. For example, the control unit can be replaced with control means or a control circuit.

Note that, in the description of the flowcharts in the present specification, the context of processing between steps is clearly indicated using expressions such as “first”, “thereafter”, and “subsequently”, but the order of processing necessary for implementing the embodiments is not uniquely determined by these expressions. That is, the order of processing in the flowcharts described in the present specification can be changed within a range without inconsistency.

According to the present disclosure, it is possible to grasp the presence or absence of a thermally denatured region outside a region of interest.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical device comprising a processor comprising hardware, the processor being configured to
acquire setting information in which a region of interest is set for a biological tissue and thermal denaturation information regarding a thermally denatured region in which thermal denaturation has occurred by heat treatment for the biological tissue,
determine whether or not there is the thermally denatured region outside the region of interest based on the setting information and the thermal denaturation information, and
output support information indicating that there is the thermally denatured region outside the region of interest when it is determined that there is the thermally denatured region outside the region of interest.

2. The medical device according to claim 1, wherein
the processor is further configured to acquire a first image obtained by imaging the biological tissue, and
the first image includes the setting information.

3. The medical device according to claim 2, wherein
the first image is a special light image generated based on an imaging signal generated by imaging return light obtained by irradiating the biological tissue with special light,
the setting information is feature data included in the special light image, and
the processor is further configured to set the region of interest based on the feature data included in the special light image.

4. The medical device according to claim 2, wherein
the first image is a white light image generated based on an imaging signal generated by imaging return light generated by irradiating the biological tissue with white light, and
the processor is further configured to
perform machine learning by using training data in which a plurality of images and annotation information added with the region of interest included in each of the plurality of images are associated with each other,
acquire, as the setting information, position information output by a learning model that uses the white light image as input data and outputs a position of the region of interest in the white light image as output data, and
set the region of interest based on the setting information.

5. The medical device according to claim 2, wherein
the first image is a white light image generated based on an imaging signal generated by imaging return light generated by irradiating the biological tissue with white light, and
the processor is further configured to
acquire, as the setting information, instruction information for instructing an annotation input from an outside for the white light image, and
set the region of interest based on the setting information.

6. The medical device according to claim 1, wherein
the processor is further configured to set the thermally denatured region according to instruction information for instructing an annotation input from an outside.

7. The medical device according to claim 1, wherein
the processor is further configured to acquire the thermal denaturation information from a second image obtained by imaging the biological tissue.

8. The medical device according to claim 7, wherein
the second image is a fluorescence image generated based on an imaging signal generated by imaging light emitted from the thermally denatured region by irradiating the biological tissue with excitation light, and
the thermally denatured region is a region that emits fluorescence.

9. The medical device according to claim 8, wherein
the processor is further configured to
determine whether or not a signal value is a predetermined threshold or more for each pixel forming the fluorescence image, and
specify a pixel with the signal value being the predetermined threshold or more as the thermally denatured region.

10. The medical device according to claim 8, wherein
the processor is further configured to
acquire a reference image based on an imaging signal obtained by imaging the biological tissue before the heat treatment, and
specify the thermally denatured region based on the reference image and the second image.

11. The medical device according to claim 1, wherein
the processor is further configured to
generate a display image indicating that there is the thermally denatured region outside the region of interest, and
output the display image as the support information.

12. The medical device according to claim 1, wherein
the processor is further configured to
generate a display image in which the thermally denatured region outside the region of interest and the thermally denatured region in the region of interest are identifiable, and
output the display image as the support information.

13. The medical device according to claim 12, wherein
the processor is further configured to
acquire a white light image generated based on an imaging signal generated by imaging return light by irradiating the biological tissue with white light,
generate the display image by identifiably superimposing the thermally denatured region outside the region of interest and the thermally denatured region in the region of interest on the white light image, and
output the display image as the support information.

14. The medical device according to claim 1, wherein
the processor is further configured to
generate a display image in which the region of interest and the thermally denatured region are identifiable, and
output the display image as the support information.

15. The medical device according to claim 1, wherein
the processor is further configured to output position information of the thermally denatured region outside the region of interest as the support information to a projector configured to project information onto the biological tissue.

16. A medical system comprising:
a light source device; an imaging device; and a medical device, wherein
the light source device includes
a special light source configured to generate special light for a biological tissue; and
an excitation light source configured to generate excitation light that excites advanced glycation end products generated by performing heat treatment on the biological tissue,
the imaging device includes an imaging element configured to generate an imaging signal by imaging return light or light emitted from the biological tissue irradiated with the special light or the excitation light,
the medical device includes a processor, and
the processor is further configured to
acquire setting information in which a region of interest is set for the biological tissue and thermal denaturation information regarding a thermally denatured region in which thermal denaturation has occurred by the heat treatment for the biological tissue,
determine whether or not there is the thermally denatured region outside the region of interest based on the setting information and the thermal denaturation information, and
output support information indicating that there is the thermally denatured region outside the region of interest when it is determined that there is the thermally denatured region outside the region of interest.

17. A learning device comprising a processor comprising hardware, the processor being configured to
generate a trained model by performing machine learning using training data in which a plurality of fluorescence images generated based on an imaging signal generated by imaging light emitted from a thermally denatured region by irradiating a biological tissue with excitation light and a plurality of white light images generated based on an imaging signal generated by imaging return light by irradiating the biological tissue with white light are input data, and support information indicating that there is a thermally denatured region included in the fluorescence image outside a region of interest included in each of the plurality of white light images is output data.

18. A method of operating a medical device including a processor, the method comprising:
acquiring, by the processor, setting information in which a region of interest is set for a biological tissue and thermal denaturation information regarding a thermally denatured region in which thermal denaturation has occurred by heat treatment for the biological tissue;
determining, by the processor, whether or not there is a thermally denatured region outside the region of interest based on the setting information and the thermal denaturation information; and
outputting, by the processor, support information indicating that there is a thermally denatured region outside the region of interest when it is determined that there is the thermally denatured region outside the region of interest.

19. A non-transitory computer-readable recording medium with an executable program stored thereon, the program causing the processor of a medical device to execute:
acquiring setting information in which a region of interest is set for a biological tissue and thermal denaturation information regarding a thermally denatured region in which thermal denaturation has occurred by heat treatment for the biological tissue;
determining whether or not there is the thermally denatured region outside the region of interest based on the setting information and the thermal denaturation information; and
outputting support information indicating that there is the thermally denatured region outside the region of interest when it is determined that there is the thermally denatured region outside the region of interest.
